SEQUENCE LISTING (1 sequence)

SEQ ID NO: 1 - protein, 8 aa, Artificial Sequence
  Synthetic
FCFWKTCT

(19) United States
(12) Patent Application Publication
(10) Pub. No.: US 2023/0210763 A1
(43) Pub. Date: Jul. 6, 2023

(54) EXTRUDED DEPOT FORM FOR CONTROLLED ACTIVE SUBSTANCE RELEASE

(71) Applicant: AMW GMBH, Warngau (DE)

(72) Inventors: Elisabeth RITTER, Warngau (DE); Alexandra FEUERSINGER, Kolbermoor (DE); Lukas OLIV, Rottach-Egern (DE); Klaus RADDATZ, Königsbronn (DE); Dirk BARTH, Gräfelfing (DE)

(73) Assignee: AMW GMBH, Warngau (DE)

(21) Appl. No.: 17/753,589

(22) PCT Filed: Sep. 18, 2020
(86) PCT No.: PCT/EP2020/076155

§ 371 Date: Mar. 8, 2022

(30) Foreign Application Priority Data
Sep. 19, 2019 (DE) 10 2019 125 208.3

(51) Int. Cl. A61K 9/00; A61K 47/34; A61K 38/31; A61K 47/14

(52) U.S. Cl. CPC A61K 9/0024; A61K 47/34; A61K 38/31; A61K 47/14

(57) ABSTRACT

The invention concerns an extruded depot form for sustained active substance release, comprising at least one active substance and at least one first compound from the class of biodegradable organic polymers based on lactic acid and/or glycolic acid.

The present invention relates to an extruded depot form comprising at least one active substance and at least one first compound from the class of biodegradable organic polymers. The present invention furthermore relates to a method for the production of the extruded depot form as well as to the use of the extruded depot form.

Subcutaneous forms of administration generally comprise liquid or solid formulations which can be administered into the subcutaneous tissue by injection or surgical intervention. In this regard, solid formulations are usually administered as cylindrical polymeric rods with the active substance embedded in it. Forms of administration which dispense active substances in this manner over a period from several days to 24 months, for example, are described as depot dosage forms.

Active substances which are released from depot dosage forms do not suffer from what is known as the first-pass effect, and therefore do not pass through the digestive tract and liver, and in addition can ensure a continuous delivery of active substance over a sustained period of time. In this manner, large variations in the active substance concentration and accompanying side effects, which often arise in the case of intravenous forms of administration, for example, can be avoided. The controlled, sustained release of active substance from depot dosage forms (also termed “depot forms” below) can additionally extend the intervals between applications. Furthermore, depot dosage forms which are biodegradable no longer have to be removed after the intended application period. These properties make subcutaneous depot forms to become user-friendly medicinal products.

The rate of release and release period for the active substance from depot forms can be influenced by the additives contained in the formulation, wherein for formulations with long application periods, great demands are made as regards the biocompatibility of the ingredients in order to minimise any compromises to the health of patients.

Thus, formulations are required which have a continuous, controlled delivery over a long application period and a high biocompatibility, as well as good properties in respect of their biodegradability.

The forms of administration described in the prior art have short application periods and/or even fail to provide the required therapeutic dose of active substance to the consumer.

Thus, the objective of the present invention is to provide a biodegradable depot form for parenteral administration of active substances which permit a sustained, controlled release of active substance in a dose which is suitable for the therapy.

In accordance with the invention, this objective is achieved by means of an extruded depot form which comprises at least one active substance and at least one compound from the class of biodegradable organic polymers in accordance with claim 1, as well as by means of a method for the production of the depot form of the invention as claimed in claim 11. Furthermore, the objective is achieved by means of a composition for use as claimed in claim 14.

Thus, the present invention concerns an extruded depot form for sustained active substance release, comprising
(a) at least one active substance,
(b) at least one first compound from the class of biodegradable organic polymers based on lactic acid and/or glycolic acid.

The designation “depot form” as used here should be understood to mean a medicinal product for which the release of active substance occurs in a delayed manner over a longer time period. In accordance with the invention, the depot form is administered parenterally and preferably forms a subcutaneous repository.

The designation “biodegradable” should be understood to mean that substances contained in the formulation are degraded or eroded away in small amounts in vivo, for example by enzymatic, chemical or physical processes.

In its simplest embodiment, the depot forms in accordance with the invention comprise at least one active substance and at least one first compound from the class of biodegradable organic polymers based on lactic acid and/or glycolic acid.

In accordance with the invention, the controlled delivery of the at least one active substance from the extruded depot form can be significantly improved by the combination of a biodegradable organic polymer based on lactic acid and/or glycolic acid. In this manner, extruded depot forms in accordance with the invention advantageously exhibit a controlled delivery of active substance in a time period from one week up to a year, a high biocompatibility and good biodegradability.

When deployed subcutaneously, the extruded depot form in accordance with the invention delivers the at least one active substance from the depot form containing the active substance into the surrounding tissue, wherein preferably, a substantial portion of the active substance is taken up systemically. Insofar as the extruded depot form is provided for local therapy, a substantial proportion of the active substance is advantageously delivered to the tissue surrounding the site of application.

The absolute active substance quantity contained in the depot form in general determines the timespan over which a continuous supply of the active substance into or onto the organism is maintained. Thus, as high a load as possible for the depot form with at least one active substance is desirable when the application period for the depot form is long, i.e. several weeks up to twelve months.

An extruded depot form in accordance with the invention is preferably used for an application period of at least one week to a maximum of 12 months, preferably for an application period from one week to 6 months, in particular for an application period from 2 weeks to 3 months.

Thus, the present invention concerns medical, veterinary and/or cosmetic use of the depot form in accordance with the invention for the delivery of active substances to the bloodstream of a human or animal body.

Furthermore, the present invention concerns a method for the production of a depot form in accordance with the invention, wherein the method comprises the following steps:
(i) providing a preferably homogeneous mixture comprising at least one active substance and at least one first compound from the class of biodegradable organic polymers based on lactic acid and/or glycolic acid,
(ii) extruding the core, in particular by melt extrusion, preferably at a temperature above the melting temperature of the at least one first compound from the class of biodegradable organic polymers based on lactic acid and/or glycolic acid,
(iii) optionally, applying a coating mixture or composition to the extrudate obtained above, preferably simultaneously with step (ii),
(iv) cutting the extrudate into pieces of suitable size,
(v) optionally, rounding the pieces, in particular by spheronisation,
(vi) optionally, carrying out a sterilisation procedure and/or packaging of the depot form.

The term “providing” as used here should be understood to mean both production on-site as well as supply of a homogeneous mixture. In this regard, a homogeneous mixture may be produced by means of a suitable mixing procedure, preferably without the addition of solvents. In addition, the mixing procedure may comprise more than one step in which firstly, for example, a mixture of the biodegradable polymer and then separately therefrom, a mixture of one or more optional excipient(s) and of the at least one active substance is formed, which is mixed with the polymer and the lipid in a second step.

The preferably homogeneous mixture which is obtained in this manner is then heated to a temperature which is preferably higher than the temperature of the melting point of the compound from the class of biodegradable organic polymers based on lactic acid and/or glycolic acid employed, and finally, it is extruded by means of extrusion, in particular by means of melt extrusion.

At the same time or after extrusion of the core (i.e. of the extrudate without any coating mixture), if appropriate, a preferably homogeneous coating mixture or composition is applied which consists of at least one of the components of the depot form in accordance with the invention mentioned above in step (i). Preferably, the coating mixture is applied simultaneously with extrusion of the core.

Preferably in this regard, the components in accordance with step (i) comprise at least approximately 50% by weight of the dry weight of the depot form in accordance with the invention, preferably at least approximately 55% by weight, particularly preferably at least approximately 60% by weight, more particularly preferably at least approximately 62% by weight, of the total weight of the depot form in accordance with the invention.

In the case in which the active substance is a heat-sensitive active substance, the extrusion may advantageously be carried out at temperatures at which even heat-sensitive active substances can be processed without having a deleterious effect on them. In this case, selection of the at least one compound from the class of biodegradable organic polymers based on lactic acid and/or glycolic acid is such that its melting point is preferably below the temperature at which the active substance is thermally compromised. This is particularly important for protein active substances, nucleic acids and the like.

If production has not already been carried out under aseptic conditions, an advantageous production method may be provided with a step for sterilisation of the extruded depot form in accordance with the invention before any possible packaging step. However, a depot form in accordance with the invention may also be produced without any sterilisation procedure, or even under conditions which are not aseptic.

Furthermore, the extruded depot form in accordance with the invention may undergo a packaging procedure in which, after any possible sterilisation procedure, the depot form is packaged directly into a packaging unit. Alternatively, the extruded depot form may also be initially introduced into an applicator provided for application of the depot form in accordance with the invention and then packaged into a packaging unit together with the applicator.

Finally, the present invention comprises an extruded depot form which can be obtained by means of the method described above.

Further particularly advantageous embodiments and further developments of the invention will become apparent from the dependent claims as well as from the description below, wherein the patent claims of a specific class may also be refined by the dependent claims of another class and features from different exemplary embodiments may be combined into new exemplary embodiments.

In accordance with a preferred embodiment, the at least one first compound is selected from the class of organic polymers based on lactic acid and/or glycolic acid selected from poly(L-lactide), poly(D,L-lactide), poly(glycolide), poly(L-lactide-co-D,L-lactide), poly(L-lactide-co-glycolide), poly(D,L-lactide-co-glycolide), poly(meso-lactide), poly(D,L-lactide-co-trimethylene carbonate), poly(L-lactide-co-meso-lactide), poly(L-lactide-co-epsilon-caprolactone), poly(D,L-lactide-co-meso-lactide), poly(D,L-lactide-co-epsilon-caprolactone), poly(meso-lactide-co-glycolide), poly(meso-lactide-co-trimethylene carbonate), poly(meso-lactide-co-epsilon-caprolactone), poly(glycolide-co-trimethylene carbonate), poly(glycolide-co-epsilon-caprolactone) and/or poly(glycolide-co-caprolactone), preferably from poly(L-lactide), poly(D,L-lactide), poly(glycolide), poly(L-lactide-co-D,L-lactide), poly(L-lactide-co-glycolide), poly(D,L-lactide-co-glycolide).

Particularly preferred compound(s) from the class of organic polymers based on lactic acid and/or glycolic acid which are suitable in this regard are poly(D,L-lactide) (PLA) and poly(D,L-lactide-co-glycolide) (PLGA) which, for example, can be obtained from Evonik Industries AG (Germany) with the names R 202 H (poly(D,L-lactide)) or RG 502 H and RG 752 H (PLGA).

The designation “polylactide” (synonymous with polylactic acids, PLA) should primarily be understood to mean polymers of lactic acid. The polymers, which are optically active because of their asymmetric carbon atom, may occur in the D- or L-lactide form.

The designation “polyglycolide” (PGA) should primarily be understood to mean polymers of glycolic acid (synonymous with hydroxyacetic acid).

The designation poly(lactide-co-glycolide) (PLGA) should be understood to mean a copolymer of the monomers lactide and glycolide which can be employed in different ratios. They then form a polyester of D, L-lactic acid and glycolic acid, which is biodegradable.

The molecular weight of the organic polymer based on lactic acid and/or glycolic acid, in particular PLA or PLGA, can in principle vary within a wide range. Preferably, however, the molecular weight is at least approximately 5 kDa and/or at most approximately 100 kDa. Particularly preferably, the molecular weight is at least approximately 7 kDa and/or at most approximately 60 kDa, in particular at least approximately 9 kDa and/or at most approximately 40 kDa, more particularly preferably at least approximately 10 kDa and/or at most approximately 30 kDa.

The depot form in accordance with the invention contains at least one active substance. In a non-limiting manner, this at least one active substance may be selected from the class of antibiotics, antimicrobiotics, antimycotics, antiseptics, chemotherapeutics, cytostatics, metastasis inhibitors, antiallergics, anticoagulants, sex hormones, sex hormone inhibitors, haemostyptics, hormones, peptide hormones, fusion proteins, antidepressants, vaccines, gonadotropin-releasing hormone analogues, growth factor inhibitors, hormone mimetics, multiple sclerosis therapeutics, programmed cell death receptor 1 antagonists, neuroleptics, complement system inhibitors, vitamins, antihistamines, antibodies, antibody fragments, nucleic acids, DNA, plasmid DNA, cationic DNA complexes and RNA, siRNA, mRNA and antidiabetics.

Useful active substances include, but are not limited to, heparin, heparin derivatives, hirudine, acetyl salicylic acid, enoxaparin, liraglutide, albiglutide, dulaglutide, lixisenatide, exenatide, insulin, insulin analogues, acarbose, glatirameracetate, octreotide, pasireotide, lanreotide and/or valpreotide, desmopressin, oxytocin, zafirlukast, buserelin, somatostatin, glibenclamide, gliclazide, glimepiride, gliquidone, pioglitazone, miglitol, nateglinide, mitiglinide, repaglinide, sitagliptin, vildagliptin, dexamethasone, prednisolone, corticosterone, budesonide, oestrogen, sulfasalazine, mesalazine, risperidone, paclitaxel, 5-fluoruracil, cisplatin, vinblastine, vincristine, epothilone, endostatin, angiostatin, D-Phe-Pro-Arg-chloromethylketone, aflibercept and monoclonal antibodies such as, for example, adalimumab, aducanumab, benralizumab, bevacizumab, certolizumab, denosumab, dupilumab, efalizumab, erenumab, infliximab, ipilimumab, mepolizumab, natalizumab, nemolizumab, ocrelizumab, omalizumab, pembrolizumab, pertuzumab, ranibizumab, reslizumab, rituximab, solanezumab, tocilizumab, tralokinumab, trastuzumab, ustekinumab and vedolizumab.

Preferred extruded depot forms containing at least one active substance can be used in this regard for the treatment of acromegaly; of symptoms which are associated with gastroenteropancreatic endocrinal tumours such as, for example, carcinoids with features of carcinoid syndrome; advanced neuroendocrinal tumours; and thyroid-stimulating hormone (TSH)-secreting pituitary adenomas; cancers such as multiple myeloma, for example, mantle cell lymphoma, diffuse large cell B-cell lymphoma, acute myeloid lymphoma, follicular lymphoma, chronic lymphocyte leukaemia, breast, lung, endometrial, ovarian, stomach, cervical or prostate cancer, pancreatic carcinoma, glioblastoma, kidney carcinoma; hepatocellular carcinoma, colon carcinoma, neuroendocrinal tumours, head and neck tumours, sarcoma; tumour syndromes resulting directly or indirectly from genetic defects in tumour suppressor genes such as, for example, P53, PTEN or VHL, endometrial carcinoma, lymphangioleiomyomatosis, neurofibromatosis 1, Hippel-Lindau disease; as well as rheumatoid arthritis; spondylitis ankylosans (Morbus Bechterew); psoriasis-arthritis; psoriasis; osteoarthritis; gout; asthma; bronchitis; allergic rhinitis; chronic obstructive pulmonary disease; cystic fibrosis; chronic inflammatory intestinal diseases such as irritable bowel disease, mucous colitis, ulcerative colitis, Crohn's disease, Huntington's chorea; gastritis; oesophagitis; hepatitis; pancreatitis; nephritis; lupus erythematodes; atherosclerosis; restenosis following angioplasty; left ventricular hypertrophy; myocardial infarction; stroke; ischaemic damage to the heart, lungs, intestines, kidneys, liver, pancreas, spleen and brain; acute or chronic organ transplant rejection; macular degeneration; diabetic macular oedema; hyposomatotropism; anaemia; fertility disorders; obesity; pubertas praecox; endometriosis; mastodynia; Tourette's syndrome; depression; personality disorders; compulsive disorders; ADHS in children; irritability in foetal alcohol syndrome and autism; delusions; hallucinations; epilepsy; Alzheimer's disease; Parkinson's disease; paroxysmal nocturnal haemoglobinuria; as a sedative; for gender reassignment procedures; multiple sclerosis and diabetes.

In accordance with a preferred embodiment, an advantageous extruded depot form contains at least one active substance from the class of peptide hormones. Particularly preferably, the at least one active substance is selected from octreotide, pasireotide, lanreotide and/or valpreotide. In particular, the at least one active substance is selected from octreotide.

Particularly preferably, the at least one active substance is selected from nucleic acids, preferably from DNA, plasmid DNA, cationic DNA complexes and/or RNA, siRNA and/or mRNA. In particular, the at least one active substance is selected from RNA, siRNA and/or mRNA, particularly preferably from self-replicating RNA and/or mRNA. A self-replicating RNA of this type is known in the art and may, for example, be obtained from viruses.

A preferred (self-replicating) RNA or mRNA may preferably be used as a vaccine and/or cancer therapy. In this regard, the RNA or mRNA codes for a desired antigen, like a pathogen. The vaccines therefore, for example, also stimulate immune reactions against tumour-associated antigens and stimulate defence cells to combat the cancer cells.

A further field of application is the use of RNA and/or mRNA as an active substance in the advantageous depot form for the formation of endogenous proteins and/or enzymes. In this manner, non-functional endogenous proteins and enzymes can be replaced by RNA and/or mRNA which code for a corresponding functional protein or enzyme by means of these RNA or mRNA-coded functional proteins or enzymes.

Preferably, the delivery of RNA or mRNA to or on a specific organ or tissue is of relevance. In particular, the delivery of RNA and/or mRNA from the depot form is carried out into the eye. Particularly preferably, an application of this type is used for the therapy of macular degeneration and/or glaucoma, of diabetes-related eye diseases as well as oncologically-related eye diseases.

Advantageously, the immune-stimulating or therapeutic properties or an effect of the RNA or mRNA can be enhanced by the addition of an adjuvant. Adjuvants of this type are known in the art. Thus, it has been shown that, for example, the RNA vaccine, which is already highly versatile, is more effective when it is formulated in a cationic oil-in-water nanoemulsion based on squalene and polysorbate (Tween 80) and sorbitan trioleate (Span 85), sodium citrate and citric acid such as, for example, Adjuvans MF59 (Novartis). With mRNA, for example, the effect can be reinforced by combining the molecules with TriMix (mRNAs which code for three proteins which activate the immune system, namely caTLR4, CD40L and CD70).

In accordance with a further preferred embodiment, the depot form comprises at least one second compound from the class of lipids.

In this regard, the dry weight of the at least one first compound from the class of biodegradable organic polymers based on lactic acid and/or glycolic acid and the at least one second compound from the class of lipids has a proportion of more than approximately 50% by weight, preferably more than approximately 55% by weight, particularly preferably more than approximately 60% by weight, more particularly preferably at least approximately 62% by weight, with respect to the total weight of the depot form.

The dry weight of the at least one compound from the class of the biodegradable polymers and of the at least one compound from the class of lipids, however, has a maximum proportion of approximately 99% by weight, preferably a maximum of approximately 97.5% by weight, particularly preferably a maximum of approximately 95% by weight, more particularly preferably of approximately 90% by weight, with respect to the total weight of the depot form.

The designation “approximately” means that a specific measured value such as, for example, a content in the composition, may deviate from the given measured value within the measurement tolerances of a suitable measuring method.

The “dry weight” of the depot form here is the weight of a formulation which is ready for administration, which has no water or a negligible amount of water, in particular less than approximately 3% by weight.

The total content of the at least one compound from the class of the biodegradable polymers therefore comprises the content of all compounds from the class of biodegradable polymers and from the class of lipids in the depot form containing the active substance.

In a depot form in accordance with the invention, the at least one second compound from the class of lipids has a melting point which is above room temperature, i.e. a temperature of approximately 25° C. Preferably the melting point of the at least one lipid is over approximately 40° C., particularly preferably over approximately 50° C., in particular over approximately 60° C., more particularly preferably over approximately 70° C. However, the melting point of the lipid in the depot form in accordance with the invention is not over 100° C.

In accordance with a further preferred embodiment, the lipid contained in the extruded depot form has a melting point of at least approximately 60° C. and/or at most approximately 80° C. Particularly preferably, the melting point of the lipid is at least approximately 62° C., in particular at least approximately 69° C. The particularly preferred melting point is at most approximately 77° C., in particular approximately 73° C.

In accordance with a further preferred embodiment, the at least one lipid is selected from mono-, di- and/or triglycerides, for example esters of glycerine with saturated and/or unsaturated fatty acids with a length of at least 5 and/or at most 26 carbon atoms, phosphatidic acids, lecithin, phosphatidyl ethanolamine, phosphatidyl inositol, phosphatidyl serine, diphosphatidyl glycerine, ceramides, cerebrosides, gangliosides, sphingophospholipids, sphingomyelins, sphingosulphatides, glycosphingosides, acylamino sugars, acylamino sugar glycans, acyltrehaloses, acyltrehalose glycans, sorbitol fatty acid esters, squalene, steroids, polyketides, sterol lipids, prenol lipids, cholesterol, hard fats, waxes, and salts and derivatives thereof.

Examples of preferred lipids are hard fats which, for example, consist of a mixture of mono-, di- and triglycerides which can be obtained by esterification of fatty acids of natural origin with glycerine or by transesterification of fats of natural origin. Particularly preferred lipids include fatty acids with a number of C atoms of at least 12 and/or at most 22, in particular at least 18 and/or at most 22. A more particularly preferred lipid is selected from glyceryl tristearate. Hard fats of this type have been described in the Pharmacopoea Europaea (Ph. Eur. 8th edition, basic edition 2014) and may, for example, be selected from those with the designation Dynasan 112, Dynasan 116 and/or Dynasan 118. These can be obtained, for example, from 101 Oleo GmbH (Germany). In the case in which heat-sensitive active substances are to be used, lipids with a melting point of less than 50° C. are preferred, such as Witepsol E85, Witepsol H5, Witepsol H12, Witepsol H37 and/or Witepsol H15, which, for example, can be obtained from 101 Oleo GmbH (Germany).

Furthermore, extruded depot forms may also comprise more than one compound from the class of lipids. Preferably, however, the extruded depot forms comprise only one compound from the class of lipids.

In accordance with a preferred embodiment, the ratio of the at least one first compound from the class of polymers and the at least one second compound from the class of lipids is at most approximately 25:1, preferably at most approximately 20:1, particularly preferably at most approximately 19:1. However, the ratio of the at least one first compound from the class of polymers and of the at least one second compound from the class of lipids is at least approximately 5:1, preferably at least approximately 10:1, particularly preferably at least approximately 12:1.

Exemplary combinations of at least one compound from the class of organic polymers based on lactic acid and/or glycolic acid and at least one compound from the class of lipids are summarised in Table 1.

TABLE 1
Exemplary compositions for depot forms
in accordance with the invention.

| Organic polymer based on | | |
|---|---|---|
| lactic acid and/or glycolic | | |
| acid | Lipid | Weight ratio |
| 32.5% by weight polylactic | — | n.a. |
| acid-co-glycolic acid polymer | | |
| and 32.5% by weight | | |
| polylactic acid | | |
| 31.5% by weight polylactic | 3.3% by weight triglyceride | 19:1 |
| acid-co-glycolic acid polymer | | |
| and 31.5% by weight | | |
| polylactic acid | | |
| 30.0% by weight polylactic | 3.5% by weight triglyceride | 17:1 |
| acid-co-glycolic acid polymer | | |
| and 30.0% by weight | | |
| polylactic acid | | |
| 57.0% by weight polylactic | 10.0% by weight glycerol | 6:1 |
| acid-co-glycolic acid polymer | dibehenate | |
| 28.5% by weight polylactic | 3.0% by weight triglyceride | 19:1 |
| acid-co-glycolic acid polymer | | |
| and 28.5% by weight | | |
| polylactic acid | | |
| 38.0% by weight polylactic | 3.0% by weight triglyceride | 21:1 |
| acid-co-glycolic acid polymer | | |
| and 26.0% by weight | | |
| polylactic acid | | |

n.a. = not applicable.

Further preferred depot forms are used for the treatment of acromegaly; of symptoms which are associated with (functionally active) gastroenteropancreatic endocrinal tumours such as, for example, carcinoids with features of carcinoid syndrome; advanced neuroendocrinal tumours; and thyroid-stimulating hormone (TSH)-secreting pituitary adenomas.

in this regard, the particularly preferred active substance octreotide may advantageously comprise a polypeptide produced from amino acids with the following sequence:

FCFWKTCT (Phe-Cys-Phe-Trp-Lys-Thr-Cys-Thr)

The at least one active substance may furthermore be contained in different forms in the depot form, depending on which form provides the optimum delivery properties for the active substance from the depot form. Active substances based on amino acids may in general be present as the cyclopeptide, oligopeptide or polypeptide or other pharmacologically acceptable derivatives, or as components of molecular complexes. In this regard, the amino acids may be bonded to each other via α-peptide linkages as well as via ω-peptide linkages. The at least one active substance may also be in the form of the salt, for example as the acetate, or also in the form of the free base or acid.

Furthermore, at least one of the amino acids of the amino acid-based active substances mentioned above as preferred active substances may have post-translational modifications. In this regard, these post-translational modifications advantageously do not influence the properties of the active substance, in particular as regards release and action.

In principle, the active substance content in the depot form in accordance with the invention may vary within a wide range.

In accordance with a preferred embodiment, an advantageous active substance comprises a nucleic acid, preferably from the class of DNA, plasmid DNA, cationic DNA complexes and/or RNA, siRNA, mRNA, particularly preferably from RNA, siRNA and/or mRNA, in particular from self-replicating RNA and/or mRNA, in a quantity in the depot form of at least approximately 1% by weight, preferably at least approximately 5% by weight, particularly preferably approximately 8% by weight. The quantity of an active substance of this type in the depot form is at most up to approximately 40% by weight, preferably up to approximately 30% by weight, particularly preferably up to approximately 20% by weight. In particular, an active substance of this type is present in a quantity of approximately 10% by weight and/or up to approximately 15% by weight.

An advantageous quantity of active substance, preferably of a peptide hormone, particularly preferably octreotide, pasireotide, lanreotide and/or valpreotide, in particular octreotide, is approximately 0.3% by weight to approximately 50% by weight, preferably approximately 3% by weight to approximately 45% by weight, particularly preferably approximately 4% by weight to approximately 40% by weight, in particular approximately 7% by weight to approximately 35% by weight.

For an effective therapy, the concentration of active substance which prevails in the blood of the consumer following administration of a depot form is significant. Advantageously, then, a preferred extruded depot form which preferably contains a peptide hormone, in particular octreotide, has an in vitro active substance release after 7 days of at least approximately 20% by weight and/or after 28 days of at least approximately 60% by weight, with respect to the total quantity of active substance contained in the implant.

Furthermore, extruded depot forms in accordance with the invention are suitable for cosmetic applications. In particular, an advantageous depot form may be used for cosmetic wrinkle reduction. In this regard, the composition in accordance with the invention is used for local, in particular for targeted wrinkle reduction, particularly preferably to prevent wrinkles, for firming skin and to protect against skin aging. Examples of active substances in this regard may be selected from hyaluronic acid, collagen and/or botox.

Advantageous depot forms may, moreover, contain at least one excipient which is in routine use in subcutaneous forms of application and which can influence the release of the active substance from the depot form, the stability of the active substance, the plasma half-life and/or the bioavailability of the at least one active substance. Advantageously, a preferred excipient supports the controlled delivery of the at least one active substance from the depot form. In particular, an excipient of this type contributes to prolonged sustained active substance delivery without, however, deleteriously affecting the biocompatibility. As an alternative or in addition, the addition of such an excipient could improve the stability of the active substance contained in the depot form. This is of particular importance when the depot form is provided for a prolonged sustained application of several weeks up to a year.

In this regard, substances which are used in the production of subcutaneous implants and which are physiologically innocuous may be mentioned. Advantageously, the excipients have a high biocompatibility, so that the excipients and any degradation products from the excipients are not toxic to the consumer and do not give rise to any undesirable side effects.

It has been shown that the addition of pore-forming agents can advantageously improve the delivery of the at least one active substance from the subcutaneous depot form. A pore-forming agent of this type may, for example, be selected from the group formed by hydrophilic materials such as calcium sulphate, calcium hydrogen phosphate, sugars such as, for example, glucose, lactose, fructose, mannitol, trehalose, dextrin, maltodextrin, saccharose, sorbitol, xylitol, starches or their derivatives such as, for example, hydroxyethyl starches, polyvinylpyrrolidone, polyethylene glycol (PEG) such as, for example, PEG 6000 or PEG 8000, sodium chloride, sodium citrate, citric acid, hyaluronic acids, polyvinyl alcohol, polyacrylic acid and its derivatives, polymethacrylic acid and its derivatives, polymethylmethacrylate, polystyrene, copolymers with monomers of methylmethacrylate and styrene, and mixtures thereof.

Particularly preferred pore-forming agents are trehalose and/or hydroxyethyl starch and/or polyethylene glycol which, for example, can be obtained from Clariant or Sigma-Aldrich (Austria).

The molecular weight of a pore-forming agent, in particular PEG, in this regard is preferably at least approximately 1 kDa, particularly preferably at least approximately 3 kDa, in particular at least approximately 4 kDa. A preferred molecular weight for PEG is at most approximately 10 kDa, particularly preferably at most approximately 8 kDa.

In accordance with a further preferred embodiment, an advantageous depot form may comprise excipients which inhibit any acylation of polypeptides, preferably of octreotide, pasireotide, lanreotide and/or valpreotide, particular preferably of octreotide. In this regard, divalent salts or salts of divalent metal ions, for example calcium chloride, are preferred.

In accordance with a further preferred embodiment, an excipient comprises at least one enzyme. An enzyme of this type may favourably affect the delivery of the at least one active substance from the advantageous depot form without having a deleterious effect on the stability and/or the biodegradability of the depot form thereby. Advantageously, an enzyme of this type improves the biodegradability of the depot form. Particularly preferably, an enzyme of this type is selected from the group formed by lipases. A lipase of this type may, for example, be obtained from Sigma-Aldrich.

The delivery rate for the active substance may furthermore be increased by adding a swellable polymer which is preferably selected from collagen, gelatines and their derivatives, starches and their derivatives (preferably hydroxyethyl starch, hydroxypropyl starch, carboxymethyl starch), cellulose derivatives, chitin, chitosan and their derivatives, polyamides, polyhydroxy acids, polyhydroxybutyrates, polyhydroxyvalerates, polycaprolactones and polydioxanones. This is particularly relevant for active substances for which a higher dose is necessary and/or for depot forms containing active substances with a shorter duration of application such as, for example, an application period of a few weeks to a few months. A particularly preferred swellable polymer in this regard is hydroxyethyl starch (HES) and can be obtained from Sigma Aldrich (Austria).

Preferably, the molecular weight of a swellable polymer, in particular of HES, is at least approximately 50 kDa, preferably at least approximately 70 kDa, in particular at least approximately 100 kDa, more particularly preferably at least approximately 120 kDa. The highest molecular weight of a swellable polymer, in particular of HES, is approximately 400 kDa, particularly preferably at most approximately 300 kDa, in particular at most approximately 200 kDa, more particularly preferably at most approximately 150 kDa.

The degree of substitution of HES, i.e. the ratio of the number of glucose units modified by hydroxyalkyl groups to the total number of monomer units, is at least approximately 0.1, preferably at least approximately 0.2, in particular at least approximately 0.3. The highest degree of substitution is approximately 1, preferably approximately 0.7, in particular approximately 0.5.

In addition, the depot forms in accordance with the invention may contain further usual excipients which are known to the person skilled in the art such as, for example, tocopherols, for example α-tocopherol, β-tocopherol, γ-tocopherol, δ-tocopherol and mixtures thereof (vitamin E), which in particular are used as antioxidants.

In an advantageous embodiment, an antioxidant of this type inactivates reactive oxygen species in the depot form, whereupon oxidation of the active substance is slowed down or completely prevented, and therefore improves the stability of the active substance and thus extends the shelf life of the depot form in accordance with the invention, both during storage as well as during use.

Advantageously, the content of the one or more of the preferred excipients brings about a controlled and sustained delivery of active substance from the preferably extruded depot form.

Advantageous compositions of an extruded depot form are constituted as follows:
(i) 0.1 to 50% by weight of active substance, preferably 1 to 50% by weight of active substance, in particular 2.5 to 40% by weight of active substance,
(ii) 1 to 95% by weight of an organic polymer based on lactic acid and/or glycolic acid, preferably 10 to 89% by weight of an organic polymer based on lactic acid and/or glycolic acid,
(iii) optionally, 1 to 25% by weight of trehalose, preferably 5 to 20% by weight of trehalose,
(iv) optionally, 1 to 25% by weight of PEG, preferably 5 to 20% by weight of PEG.

Particularly preferably, a composition of an extruded depot form comprises:
(i) 5 to 45% by weight of active substance, in particular 10 to 40% by weight of active substance,
(ii) 30 to 85% by weight of an organic polymer based on lactic acid and/or glycolic acid, preferably PLGA and/or PLA,
(iii) optionally, 2.5 to 18% by weight of trehalose, preferably 3 to 16% by weight of trehalose, in particular 3.5 to 15% by weight of trehalose, and
(iv) optionally, 3 to 17.5% by weight of PEG.

More particularly preferably, the components of an extruded depot form are selected from:
(i) 15 to 35% by weight of active substance,
(ii) 40 to 75% by weight of an organic polymer based on lactic acid and/or glycolic acid, preferably PLGA and/or PLA
(iii) 1 to 10% by weight of a lipid,
(iv) optionally, 5 to 10% by weight of trehalose, and
(v) optionally, 5 to 15% by weight of PEG.

More preferably, the components of an extruded depot form are selected from:
(i) 0.1 to 50% by weight of active substance, preferably 1 to 50% by weight of active substance, in particular 2.5 to 40% by weight of active substance, more particularly preferably 5 to 20% by weight of active substance,
(ii) 40 to 75% by weight of an organic polymer based on lactic acid and/or glycolic acid, preferably PLGA and/or PLA,
(iii) optionally, 1 to 10% by weight of a lipid,
(iv) optionally, 0.1 to 2% by weight of a lipase, preferably 0.2 to 1% by weight,
(v) optionally, 5 to 10% by weight of trehalose, and
(vi) optionally, 5 to 15% by weight of PEG.

In particular, an advantageous active substance content is approximately 5 to 30% by weight, a biodegradable organic polymer content is approximately 50 to 70% by weight and an optional lipid content is approximately 5% by weight.

In accordance with a further preferred embodiment, trehalose may be completely or partially replaced by HES.

As already mentioned, extruded depot forms in accordance with the invention are produced by extrusion. It has been shown that in this manner, the properties of the mixture of the at least two compounds from the class of organic polymers based on lactic acid and/or glycolic acid and from the class of lipids with the at least one active substance prepared for extrusion are improved when the active substance is admixed with the at least two compounds as a dry powder, preferably as spray dried or freeze dried powder or lyophilisate.

If active substances which are dissolved are used, prior to mixing the substances for the production of the depot form in accordance with the invention, a drying step is preferably carried out, preferably a freeze drying step.

For this, in principle, a plurality of substances may be added to the active substance in order, advantageously, to guarantee maintaining the bioactivity of the active substance. Substances of this type are described as cryoprotectors or lyoprotectors, wherein lyoprotectors in this connection act to protect the substances when drying and cryoprotectors have a similar function during freezing.

Furthermore, it has been shown that the duration of active substance release after subcutaneous application can be influenced by cooling the extrudates directly after extrusion, not to ambient temperature but, for example, to store them in a drying cabinet or incubator for a specific time at a raised temperature. In this regard, a duration in the range from approximately 0.5 to approximately 5 hours is suitable. The optional storage at raised temperature, also termed the tempering step, is essentially based here on the melting point of the at least one lipid and, for example, is in the range from approximately 40° C. to 80° C., preferably approximately 55° C. to approximately 75° C., particularly preferably approximately 65° C. to 70° C. Clearly, a preferred storage temperature will always be a function of the stability of the at least one active substance to temperature.

Preferred depot forms can, moreover, be produced by means of a rounding method, in particular spheronisation. In this regard, the cylindrical extrudate is advantageously rounded in a manner such that shapes which are formed by the extrusion, such as corners and edges, which could adversely affect the application properties, are removed. Moreover, spheronisation can be used for the production of microparticles which are then administered subcutaneously and thus also constitute a biodegradable depot form.

Advantageously, a depot form has a homogeneous coating which consists of at least one layer applied to the core and preferably defines the initial release of active substance from the depot form and guarantees therapeutic concentrations of the at least one active substance over a sustained time period. A preferred coating has a mixture of substances or a composition which is or are selected from at least one of the aforementioned components a) to c) of the depot form in accordance with the invention. Insofar as the coating contains an active substance, the quantity thereof may be the same as or different from the quantity of active substance in the core. In a particularly preferred embodiment, however, the coating is free from any active substance.

In principle, a suitable weight for an extruded depot form is in the range which is usual for a subcutaneous implant. In this regard, the weight of the extruded depot form is also dependent on the desired application period and/or site of application. A preferred weight for an extruded depot form, however, is in the range from 1 to 1000 mg, particularly preferably at least approximately 20 mg and/or at most approximately 180 mg, in particular at least approximately 50 mg and/or at most approximately 150 mg, more particularly preferably at least approximately 80 mg and/or at most approximately 120 mg.

A depot form in the context of the invention may in this regard be formed as rods, balls, cubes, ellipsoids, cuboids, cushions, cylinders, tablets, pellets, platelets or briquettes.

Depot forms in accordance with the invention are preferably of an injectable size but, if desired, may also be introduced to the site of administration by means of a surgical intervention. In this regard, preferred depot forms have a diameter of at least 0.1 to 10 mm and a length of at least 0.15 to 50 mm. In particular, depot forms have a diameter of at least 0.15 to 7.5 mm and a length of at least 0.2 to 45 mm, particularly preferably a diameter of at least 0.2 to 5 mm and a length of at least 0.3 to 40 mm.

The term “diameter” in this regard refers to the longest extent which is orthogonal to an axis of rotation which connects two points of the periphery of the body concerned. The “axis of rotation” is the straight line about which a body of rotation can be turned. The term “length” refers to a portion of the axis of rotation which is within the body or rotation.

The ratio of diameter to length of preferred depot forms is advantageously in the range from 1:30 to 10:1, preferably in the range from 1:15 to 5:1, more particularly preferably in the range from 1:13 to 1:1.

When the extruded depot forms are microparticles, the diameter of the round or almost round particles may be approximately 1 to approximately 100 μm, preferably approximately 5 to approximately 90 μm, more particularly preferably approximately 10 to approximately 80 μm.

As already explained above, a method in accordance with the invention for the production of the extruded depot form comprises mixing (a) the at least one active substance, and (b) the at least one compound from the class of organic polymers based on lactic acid and/or glycolic acid, optionally (c) the at least one compound from the class of lipids, whereupon a homogeneous powdered mixture is obtained.

The term “powdered mixture” as used in the context of the present invention should be understood here to mean a mixture of a plurality of solid components, wherein the components may have particles with a size of less than 1 nm. Furthermore, a powdered mixture may also have particles with a size in the range from 1 nm to 1 μm and/or particles with a size of more than 1 μm. If at least one of the components to be mixed is not in the solid form prior to mixing, before constituting the powdered mixture, they can be transformed into the solid state, for example by spray drying or freeze drying.

In order to apply the extruded depot form in accordance with the invention to the subcutaneous tissue, in principle, any of the application devices known to the person skilled in the art may be used. Thus, depot forms in accordance with the invention may, for example, be administered by syringes, cannulas, applicators and injectors, in particular by applicators.

Finally, the present invention also concerns a kit comprising an extruded depot form in accordance with the invention and an applicator which is suitable for application, with which the depot form can be administered subcutaneously. In this regard, extruded depot forms in accordance with the invention do not necessarily have to be sterilised prior to being received in the applicator, but may also undergo a sterilisation procedure inside the applicator. Furthermore, an applicator of this type is capable of accommodating extruded depot forms with different lengths. In this regard, clearly, depot forms which are not cylindrical in shape but which, for example, are cuboid or round or the like, may be applied.

Advantageously, an applicator of this type has a hollow needle for receiving extruded depot forms with the dimensions discussed above and a protective cap which has to be removed before application and can be re-attached after use. In this manner, the extruded depot form can advantageously be protected against external influences which could have a negative effect of some kind on the preferred use.

Further features of the invention will become apparent from the description below of exemplary embodiments made in connection with the claims as well as the figures. It should be noted that the invention is not limited to the embodiments in the described exemplary embodiments, but is determined by the scope of the accompanying patent claims. In particular, the individual features of the embodiments in accordance with the invention may be produced in a combination other than in the examples given below. The description below of some exemplary embodiments of the invention refers to the accompanying drawings, in which:

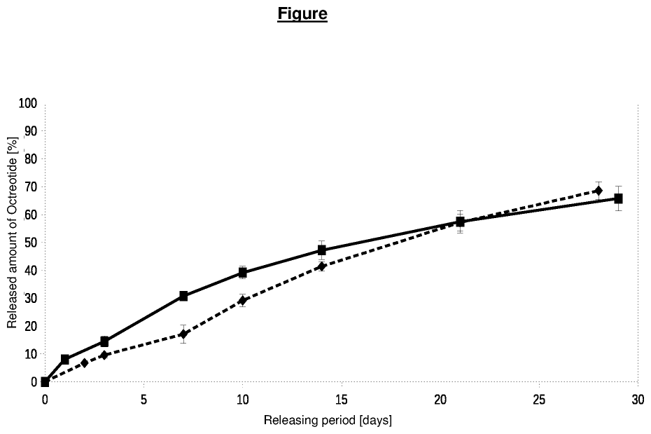

FIG. 1: shows the release profiles for an octreotide-containing depot form in accordance with the invention (in accordance with Example 2, see below) compared with an octreotide-containing depot form which is not in accordance with the invention. The depot forms have a diameter of approximately 2 mm. Solid line (example in accordance with the invention): 33% by weight octreotide, 32% by weight polylactic acid-co-glycolic acid polymer, 32% by weight polylactic acid, 3% by weight triglyceride. Dashed line (comparative example): 33% by weight octreotide; 33.5% by weight polylactic acid-co-glycolic acid polymer, 33.5% by weight polylactic acid.

### EXAMPLES

Example 1, in accordance with the invention:

In order to produce depot forms in accordance with the invention, a powdered mixture was weighed out which consisted of equal parts of 34% by weight of the polylactic acid-co-glycolic acid polymer (Resomer® RG752H, Evonik Industries, Germany), 34% by weight of polylactic acid (Resomer® R202H, Evonik Industries, Germany), and 32% by weight of octreotide lyophilisate (Bachem, Bubendorf, Switzerland). This powdered mixture was homogeneously mixed by cryogenic milling (Freezer/Mill, C3 Prozess- and Analysentechnik GmbH, Haar bei Munchen, Germany).

The subsequent extrusion was carried out by means of counter-rotating screw melt extrusion (Mini CTW, Thermo Fisher Scientific GmbH, Karlsruhe, Germany) at 85° C. to 92° C. and at a screw rotation speed of 8 rpm (revolutions per minute). The diameter of the extrudate was set at 2.0 mm with a nozzle. The extruded rod was cut into extrudates of a suitable length (in the present example to a length of 2 cm). Alternatively, the extrudate could have been shaped into microparticles by spheronisation.

Example 2, in accordance with the invention:

In order to produce depot forms in accordance with the invention, a powdered mixture was weighed out which consisted of 32% by weight of polylactic acid-co-glycolic acid polymer (Resomer® RG752H, Evonik Industries, Germany), 32% by weight of polylactic acid (Resomer® R202H, Evonik Industries, Germany), 3% by weight of a triglyceride (Dynasan 118, 101 Oleo GmbH, Hamburg, Germany) and 33% by weight of octreotide lyophilisate (Bachem, Bubendorf, Switzerland). This powdered mixture was homogeneously mixed by cryogenic milling (Freezer/Mill, C3 Prozess- and Analysentechnik GmbH, Haar bei Munchen, Germany). The subsequent extrusion was carried out by means of counter-rotating screw melt extrusion (Mini CTW, Thermo Fisher Scientific GmbH, Karlsruhe, Germany) at 85° C. to 92° C. and at a screw rotation speed of 8 rpm (revolutions per minute). The diameter of the extrudate was set at 2.0 mm with a nozzle. The extruded rod was cut into extrudates of a suitable length (in the present example to a length of 2 cm). Alternatively, the extrudate could have been shaped into microparticles by spheronisation.

Example 3, in accordance with the invention:

Production was as described in Example 1, but the composition of the depot form in accordance with the invention was supplemented with calcium chloride (Sigma Aldrich, Vienna, Austria). The powdered mixture consisted of 31.75% by weight of polylactic acid-co-glycolic acid polymer (Resomer® RG752H, Evonik Industries, Germany), 31.75% by weight of polylactic acid (Resomer® R202H, Evonik Industries, Germany), 3.5% by weight of calcium chloride and 33% by weight of octreotide lyophilisate.

Example 4, in accordance with the invention:

Production was as described in Example 1, but the composition of the depot form in accordance with the invention was supplemented with a triglyceride and trehalose (Sigma Aldrich, Vienna, Austria). The powdered mixture consisted of 30% by weight of polylactic acid-co-glycolic acid polymer (Resomer® RG752H, Evonik Industries, Germany), 30% by weight of polylactic acid (Resomer® R202H, Evonik Industries, Germany), 3.5% by weight of triglyceride (Dynasan 118, IOI Oleo GmbH, Hamburg, Germany), 3.5% by weight of trehalose and 33% by weight of octreotide lyophilisate.

Example 5, in accordance with the invention:

In order to produce depot forms in accordance with the invention, a powdered mixture was produced which consisted of 57% by weight of polylactic acid-co-glycolic acid polymer (Resomer® RG752H, Evonik Industries, Germany), 10% by weight of glycerol dibehenate (Compritol 888 ATO, Gattefosse) and 33% by weight of octreotide lyophilisate. The components of this powdered mixture were homogeneously mixed by means of cryogenic milling (Freezer/Mill, C3 Prozess- and Analysentechnik GmbH, Haar bei Munchen, Germany). The subsequent extrusion was carried out by means of co-rotating screw melt extrusion (Mini CTW, Thermo Fisher Scientific GmbH, Karlsruhe, Germany) at 83° C. to 90° C. and at a screw rotation speed of 8 rpm.

Example 6, in accordance with the invention:

In order to produce depot forms in accordance with the invention, a powdered mixture which consisted of 28.5% by weight of polylactic acid-co-glycolic acid (Resomer® RG502H, Evonik Industries, Germany), 28.5% by weight of polylactic acid (Resomer® R203H, Evonik Industries, Germany), 3% by weight of triglyceride (Dynasan 118, 101 Oleo GmbH, Hamburg, Germany) and 40% by weight of lanreotide was processed into a homogeneous mixture by means of cryogenic milling. To examine the active substance release, exemplary depot forms in accordance with the invention in the respectively suitable shape and sizes (for example cut into cylinders 1.5 to 2 cm in length and with a diameter of 1.8 mm) were initially weighed out separately.

Example 7, in accordance with the invention:

In order to produce depot forms in accordance with the invention, a powdered mixture was weighed out which consisted of 38% by weight of the polylactic acid-co-glycolic acid polymer (Resomer® RG752H, Evonik Industries, Germany), 26% by weight of polylactic acid (Resomer® R202H, Evonik Industries, Germany), 3% by weight of a triglyceride (Dynasan 118, IOI Oleo GmbH, Hamburg, Germany) and 33% by weight of octreotide lyophilisate (Bachem, Bubendorf, Switzerland). This powdered mixture was homogeneously mixed by cryogenic milling (Freezer/Mill, C3 Prozess- und Analysentechnik GmbH, Haar bei Munchen, Germany). The subsequent co-extrusion was carried out with the aid of a dual-component nozzle wherein, in addition to the actual powdered mixture, a homogeneous coating produced from 100% by weight polylactic acid-co-glycolic acid polymer (Resomer® RG752H, Evonik Industries, Germany) was extruded with the aid of a structurally identical second extruder (Mini CTW, Thermo Fisher Scientific GmbH, Karlsruhe, Germany). The diameter for the core extrudate was set at 2.0 mm and the layer thickness of the coating was set to 0.1 mm with the aid of the dual-component nozzle. The extruded rod was then cut into extrudates of a suitable length.

### Comparative Example

For the production of a depot form which acted as a comparative example, a powdered mixture was produced which consisted of 33.5% by weight of the polylactic acid-co-glycolic acid polymer (Resomer® RG752H, Evonik Industries, Germany), 33.5% by weight of polylactic acid (Resomer® R202H, Evonik Industries, Germany) and 33% by weight of octreotide lyophilisate (Bachem, Bubendorf, Switzerland). This powdered mixture was homogeneously mixed by cryogenic milling (Freezer/Mill, C3 Prozess- und Analysentechnik GmbH, Haar bei Munchen, Germany).

The subsequent extrusion was carried out by means of counter-rotating screw melt extrusion (Mini CTW, Thermo Fisher Scientific GmbH, Karlsruhe, Germany) at 85° C. to 92° C. and at a screw rotation speed of 5-15 rpm (revolutions per minute). The diameter of the extrudate was set at 2.0 mm with a nozzle. The extruded rod was cut into extrudates of a suitable length (in the present example to a length of 2.0 cm).

Determination of the in vitro release of the active substance from the depot forms (Examples 1 to 7 as well as comparative example):

In order to investigate the in vitro release of the active substance from the depot forms, the depot forms (in accordance with Examples 1-6) were added to release cells and supplemented with 50 mL of release medium (disodium hydrogen phosphate, pH 7.4). The depot forms were then placed in an incubating shaker (IKA®-Werke GmbH & Co. KG, Germany) at 37° C. for the desired application period.

At the respective sampling times, approximately 1 mL of sample solution was removed with a disposable pipette and placed directly into a HPLC column. After each sampling time, the release medium was replaced in its entirety.

Example 8, in accordance with the invention:

In order to produce depot forms in accordance with the invention, a powdered mixture was weighed out which consisted of 44% by weight of the polylactic acid-co-glycolic acid polymer (Resomer® RG752H, Evonik Industries, Germany), 44% by weight of polylactic acid (Resomer® R202H, Evonik Industries, Germany) and 12% by weight of mRNA (CleanCap® EGFP mRNA, TriLink Biotechnologies, Inc. USA).

The subsequent extrusion was carried out by means of co-rotating screw melt extrusion (Mini Extruder ZE-5, Three-Tec GmbH, Birnen, Switzerland) at 85° C. to 90° C. and at a screw rotation speed of 10 rpm (revolutions per minute). The diameter for the extrudate was set to 0.5 mm with a nozzle. The extruded rod was cut into extrudates of a suitable length (in the present example to a length of 0.5 cm).

The expression (assay of GFP activity) was monitored with the aid of the GFP Assay System (Arbor Assays LLC, USA). Here, the following protocol was used: the depot form in accordance with the invention was added to a human cell culture. The release from the depot form and the activity of the coded protein were detected by the fluorescence assay. Upon excitation with light at a wavelength of 395 nm, cells which have taken up the mRNA and express the GFP gene emit measurable light at a wavelength of 509 nm. In cell cultures which had received a negative control (without mRNA), no substantial fluorescence could be measured.

Example 9, in accordance with the invention:

Production was as described in Example 1, but the composition of the depot form in accordance with the invention was supplemented with glycerol dibehenate. The powdered mixture consisted of 40% by weight of polylactic acid-co-glycolic acid polymer (Resomer® RG752H, Evonik Industries, Germany), 40% by weight of polylactic acid (Resomer® R202H, Evonik Industries, Germany), 5% by weight of triglyceride (Dynasan 118, 101 Oleo GmbH, Hamburg, Germany), 4.5% by weight of glycerol dibehenate (Compritol 888 ATO, Gattefosse), 10% by weight of mRNA (CleanCap® EGFP mRNA, TriLink Biotechnologies, Inc. USA) and 0.5% by weight of lipase (triacylglycerol lipase from Pseudomonas sp., Merck KGaA, Darmstadt). The expression was assayed in the same manner as in Example 8.

Example 10, in accordance with the invention:

In order to produce depot forms in accordance with the invention, a powdered mixture was weighed out which consisted of 86% by weight of the polylactic acid-co-glycolic acid polymer (Resomer® RG752H, Evonik Industries, Germany) and 14% by weight of mRNA (CleanCap® FLuc mRNA, TriLink Biotechnologies, Inc. USA). This powdered mixture was homogeneously mixed by cryogenic milling (Freezer/Mill, C3 Prozess- and Analysentechnik GmbH, Haar bei Munchen, Germany). The subsequent extrusion was carried out by means of co-rotating screw melt extrusion (Mini Extruder ZE-5, Three-Tec GmbH, Birnen, Switzerland) at 83° C. to 92° C. and at a screw rotation speed of 10 rpm.

The expression (assay of luciferase activity) was monitored with the aid of the Luciferase Assay System E1500 (Promega Corporation, USA). Here, the following protocol was used: the depot form in accordance with the invention was added to a human cell culture. The release from the depot form and the activity of the coded protein were detected by the luciferase assay. Upon adding the substrate (luciferin), cells which have taken up the mRNA and express the luciferase enzyme emit measurable light. In cell cultures which had received a negative control (without mRNA), no substantial fluorescence could be measured.

1. An extruded depot form, comprising:
at least one active substance; and
at least one biodegradable organic polymer comprising at least one monomer selected from the group consisting of lactic acid and glycolic acid.

2. The extruded depot form of claim 1, wherein the at least one biodegradable polymer is at least one selected from the group consisting of poly(L-lactide), poly(D,L-lactide), poly(glycolide), poly(L-lactide-co-D,L-lactide), poly(L-lactide-co-glycolide), poly(D,L-lactide-co-glycolide), poly(meso-lactide), poly(D,L-lactide-co-trimethylene carbonate), poly(L-lactide-co-meso-lactide), poly(L-lactide-co-epsilon-caprolactone), poly(D,L-lactide-co-meso-lactide), poly(D,L-lactide-co-epsilon-caprolactone), poly(meso-lactide-co-glycolide), poly(meso-lactide-co-trimethylene carbonate), poly(meso-lactide-co-epsilon-caprolactone), poly(glycolide-co-trimethylene carbonate), poly(glycolide-co-epsilon-caprolactone) and poly(glycolide-co-caprolactone).

3. The extruded depot form of claim 1, wherein the at least one active substance is at least one selected from the group consisting of DNA, plasmid DNA, cationic DNA complex, RNA, siRNA and mRNA.

4. The extruded depot form of claim 1, wherein the at least one active substance is at least one selected from the group consisting of a peptide hormone, a growth hormone, a VEGF inhibitor, an antidiabetic, a gonadotropin-releasing hormone analogue (GnRH analogue), an antirheumatic, a breast cancer therapeutic, an MS (multiple sclerosis) therapeutic, a programmed cell death receptor-1 (PD-1) antagonist, a protein medicinal product against paroxysmal nocturnal haemoglobulinuria, and a neuroleptic.

5. The extruded depot form of claim 1, wherein the depot form comprises at least one lipid.

6. The extruded depot form of claim 5, wherein the at least one active substance, the at least one biodegradable organic polymer, and the at least one lipid comprises at least 50% by weight of the dry weight of the depot form.

7. The extruded depot form of claim 1, further comprising one or more excipients selected from the group consisting of an enzyme, polyethylene oxide, a salt with divalent metal ions, a disacharide, an oligosaccharide, and a polysaccharide.

8. The extruded depot form of claim 1, wherein the extruded depot form comprises a homogeneous core coating comprising a homogeneous composition of the at least one active substance and the at least one biodegradable organic polymer.

9. The extruded depot form of claim 1, wherein the extruded depot form has a length of 0.1 cm to 5 cm.

10. The extruded depot form of claim 1, wherein the extruded depot form has a ratio of diameter to length of 1:30 to 10:1.

11. A method for production of an extruded depot form of claim 1, comprising:
providing a homogeneous mixture of the at least one active substance and the at least one biodegradable organic polymer,
extruding the mixture at a temperature above the melting temperature of the at least one biodegradable organic polymer to obtain an extrudate,
optionally, applying a coating mixture or composition to the extrudate,
cutting the extrudate into pieces,
optionally, rounding the pieces,
optionally, sterilizing and/or packaging the depot form.

12. An extruded depot form obtained in accordance with the method of claim 11.

13. The extruded depot form of claim 1, wherein the in vitro release of the peptide hormone is at least 20% by weight after 7 days and/or is at least 60% by weight after 28 days.

14. A medical, veterinary or cosmetic composition, comprising the extruded extruded depot form claim 1, for medical, veterinary or cosmetic application.

15. A method of treating a condition, comprising providing a pharmaceutically effective amount of the extruded depot form of claim 1 to a patient, the condition comprising at least one selected from the group consisting of acromegaly; gastroenteropancreatic endocrinal tumours; advanced neuroendocrinal tumours; thyroid-stimulating hormone (TSH)-secreting pituitary adenomas; vaccination and/or cancer therapy; the non-functional expression of proteins and/or enzymes; macular degeneration and/or glaucoma, diabetes-related eye disease and oncologically-related eye disease.

16. The extruded depot form of claim 4, wherein the at least one active substance is at least one selected from the group consisting of octreotide, pasireotide, lanreotide, valpreotide, somatotropin, erythropoetin, bevacizumab, ranibizumab, exenatide, goserelin, leuprorelin, infliximab, etanercept, trastuzumab, natalizumab, pembrolizumab, eculizumab, and risperidone.

17. The extruded depot form of claim 7, wherein the one or more excipients is selected from the group consisting of a lipase, polyethylene glycol, calcium chloride, trehalose, a cyclodextrin, and cellulose.

18. The method of claim 11, wherein the coating composition is applied to the extrudate simultaneously with extruding the core to obtain the extrudate.
